(19) United States
(12) Patent Application Publication
(10) Pub. No.: US 2011/0288652 A1
(43) Pub. Date: Nov. 24, 2011

(54) MATERIALS AND METHODS FOR TREATING CRITICALLY SIZED DEFECTS IN MOUSE BONE

(75) Inventors: Melissa A. Kacena, Zionsville, IN (US); Tien-Min Gabriel Chu, Carmel, IN (US); Daniel Alge, Longmont, CO (US)

(73) Assignee: Indiana University Research & Technology Corporation, Indianapolis, IN (US)

(21) Appl. No.: 13/112,820

(22) Filed: May 20, 2011

Related U.S. Application Data
(60) Provisional application No. 61/346,706, filed on May 20, 2010.

(51) Int. Cl. A61F 2/28

(52) U.S. Cl. 623/23.61

(57) ABSTRACT

Scaffolds equipped to include biologically active reagents such as proteins sized and engineered specifically for use in a mouse were made and implanted into mice. The scaffold was specifically designed for use in a mouse femoral critical sized defect Scaffolds. Special care was taken when casting these scaffolds to ensure that the material used to form the body of the scaffold did not include defects such as a large number of macroscopic holes that could compromise the structural integrity of these very small devices. Some of these scaffolds include a hollow center that accommodate a rod that ran the longitudinal length of the scaffold extending into the adjacent bone to anchor the scaffold to the bone. Scaffold also include at least one channel that extended from the outside of the structure towards its center and could be hold a liquid or paste comprising for example exogenous BMP-2.

### PRIORITY CLAIM

This application claims the benefit of U.S. provisional patent application No. 61/346,706 filed on May 20, 2010 which is hereby incorporated by reference in its entirety.

### FIELD OF THE INVENTION

Aspects of the invention relate materials and methods for creating a scaffold that adapted to treat a specific bone defect; the method may include features the delivery of proteins to the scaffold and/or healing bone, in for example a very small mammal such as mouse.

### BACKGROUND

Segmental defects in mammal bone are often difficult to treat. Treating such defects may require multiple-phase surgery to achieve adequate union and to regain function. The causes for these large segmental defects in bone include the removal of tumors, massive trauma, congenital malformations, or nonunion of fractures. In the event that removal is necessary, the stage of the tumor generally determines the surgical margins of removal, which in turn determines the magnitude of the defect. Treatment for malignant tumors, including but not limited to, osteosarcoma, Ewing's tumors, and metastatic tumors often include initially introducing cytotoxic drugs for chemotherapy prior to tumor removal surgery. The chemotherapy allows the prevention of spreading of the tumor so that only the segment of bone with the tumor itself may be removed as an alternative to amputation. The result is often a bone gap and existing bone having a compromised regenerative capacity caused by exposure to radiation and/or chemotherapeutic agents. In the event of an extreme high-energy trauma to a bone, a segmental defect may occur due to the large extent of fragmentation of the bone. To ensure union of the two ends of the defect, a support is needed for cells to proliferate across the gap.

Another common cause of segmental defects in bone is nonunion. Non-union occurs in bone gap defects when bone fails to mend due to factor such as excessive motion, gap at the fracture site, poor nutrition, significant commutation and the like. A hypertrophic non-union occurs when increased bone formation leads to an adequate healing process with lack of mechanical stability. Atrophic non-union, however, occurs when bony resorption at the fracture site and vascular compromise leads to fibrous tissue occupying the fracture gap. When a non-union has a lack of callus due to avascularity, appropriate immobilization, removal of the atrophic bone, and grafting of bone substitute material may be needed to ensure proper healing.

There is an ongoing need to treat defects in mammalian bone caused by disease, mutation, injury and the like. Given how common bone defects are and how difficult many of them are to treat there in an immediate need for effective methods to treat theses defects. Not only is there a need for materials and methods to treat bone defects and disease there is also a need for better tools to conduct research in this area. Some aspects of the invention disclosed herein seek to address this need.

### SUMMARY OF THE INVENTION

Some aspects of the invention include implantable scaffolds designed and sized for for treating critical sized bone gaps in mice. Some of these scaffolds comprise: a biodegradable scaffold; said scaffold having a body, the body having sides; a first distal end and a second distal end, and a center wherein the center accommodates or includes an anchoring member that extends beyond both the first and the second distal ends of the body of the scaffold and wherein the scaffold is sized to fit into a critical sized gap in a mouse femur; and at least one bio-reactive compound that promotes bone growth and or healing, wherein the compound is associated with the biodegradable scaffold.

In some embodiments the anchoring member in the scaffold is a rod or wire (composed of metal, plastic or other material of sufficient strength and biocompatability. In some embodiments of the invention the anchoring member is substantially comprised of a biodegradable material.

In some embodiments of the invention the body of the scaffold is sized such that the first distal end and the second distal end of the scaffolding are in contact with surfaces of mouse femur bracketing a critical sized gap in a mouse femur when the scaffold is implanted with a gap in a mouse femur. In some embodiment the scaffold is attached to the bone by use of bone cement. In some embodiment the bone cement used to attach the scaffold may include a bio-reactive compounds such as BMP.

In some embodiments the scaffold further includes at least one channel, the channel being located in a plane that intersects the center of the scaffold. In some embodiments this channel may be of substantially uniform diameter over its entire length and in some embodiment the channel may be irregular, not circular or is in the shape of a funnel. In some embodiments the channel may extend from the center of the scaffold to at least one hole in the side of the scaffold. In some embodiment of the invention at least one channel in the scaffold may include at least one compound that promotes bone growth and or bone health such as BMP, an antibiotic or BMP-2. In some embodiment the bio-reactive compound is included in a formulation that includes dicalcium phosphate dihydrate. And in some embodiment at least one channel in said scaffold is packed with a formulation dicalcium phosphate dihydrate that includes BMP.

In some embodiments of the invention the body of the scaffold is substantially comprised of N-vinyl pyrrolidinone and β-tricalcium phosphate. In some embodiments the body of the scaffold is free of macroscopic voids with the sides of the scaffold comprising the body of the scaffold and surrounding the center of the scaffold. The may scaffold may be is in the shape of a cylinder and wherein the diameter of the cylinder is less than or equal to about 0.6 mm. In some embodiments that scaffold may be in shape other than that of a cylinder.

Still another aspect of the invention is a method of manufacturing a biodegradable scaffold, comprising the steps of: providing a first paste that include free radial initiator benzoyl peroxide; supplying a second paste that includes N,N-dimethyl-p-toluidine; and mixing the first and the second paste and exposing the mixture to a vacuum for at least 30 seconds.

### BRIEF DESCRIPTION OF THE FIGURES

FIG. 1A. Dimensions of a representative Mouse femoral defect scaffold.

FIG. 1B. Macroscopic view of a representative Mouse femoral defect scaffold.

FIG. 2 A. A schematic illustrating in vivo drug delivery and bone healing scaffolding specifically designed for use in a mouse.

FIG. 2 B. A schematic illustrating a representative scaffold shown with DCPD cement carrying a drug or other biologically active compound.

FIG. 3. X-ray images of mouse femurs taken over time after treatment with two different scaffolds.

FIG. 4. Illustration of BMP scaffold that include BMP and is placed in a rat femur segmental defect stabilized with an intrameullary pin.

FIG. 5. Representative serial radiological images of segmental defects in the rat BMP and control groups taken post-operatively at one, three, six, 12 and 15 weeks.

FIG. 6 A. Representative histological images of segmental defects in a femur taken from a control rat (A) at six weeks post-operative.

FIG. 6 B. Representative histological images of segmental defects in a rat femur treated with a scaffold and rhBMP image taken six weeks post-operative.

FIG. 6 C. Representative histological images of segmental defects in the rat rhBMP group image taken six weeks post-operative (enlargement of FIG. 6B).

FIG. 6 D. Representative histological images of segmental defects in the rat rhBMP group fifteen weeks post-operative.

FIG. 7 A. Representative external and cut-away 3D reconstructed microCT images of segmental defects in a rat femur from the control group taken six weeks post-operative.

FIG. 7 B. Representative external and cut-away 3D reconstructed microCT images of segmental defects in a rat femur image taken from the group treated with BMP taken six weeks post-operative.

FIG. 7 C. Representative external and cut-away 3D reconstructed microCT images of segmental defects in a rat femur in the BMP group imaged at week 15 weeks post-operative.

FIG. 8. Photograph showing a step in the procedure implanting a scaffold in a mouse femur.

FIG. 9. Representative histological composite image of callus area surrounding an untreated control mouse scaffold 15 weeks post-operative.

FIG. 10. Representative histological composite image of callus area surrounding a BPM-2 treated mouse scaffold 15 weeks post-operative.

### DESCRIPTION

For the purposes of promoting an understanding of the principles of the novel technology, reference will now be made to the preferred embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates are within the scope of this disclosure and what it claims.

Unless stated or clearly implied otherwise the term “about” as used herein refers to a range of value of plus or minus 10 percent; e.g. about 1.0 includes the range of values encompassing 0.9 to 1.1.

Current treatment options to regenerate segmental defects include autografts, allografts, and distraction osteogenesis. Bone replacement grafts taken from the patient under treatment are known as autografts. Both cortical and cancellous bones may be used as autografts. Some of the main advantages of autografts are that they provide a structural framework for bone formation and supply factors that stimulate bone formation. Further, autografts tend to incorporate into the surrounding tissue more quickly than allografts. Disadvantages to autografts include the limited number of places from which a graft can be taken, as well as the potential of donor site morbidity.

One alternative to autografts are allografts—bone grafts obtained from a donor other than the patient being treated. While allografts pose no risk of donor site morbidity, allografts do introduce the possibility of the disease transmission. Further, allografts include the potential of tissue rejection or a negative immune response, both of which are very unlikely in using an autograft. A decrease in the immunogenicity of bone allografts may result in better healing and mechanical strength, and while histocompatibility matching has been examined, it has not been clinically proven. An effective way of reducing the immunogenicity of allografts is to alter the individual grafts. Some methods of altering the allografts to prevent transmission of disease include sterile harvesting techniques, deep freezing, low level radiation, and freeze drying. While these techniques reduce the likelihood of disease transmission, they can reduce the osteoconductivity of allografts. Therefore, a method of conducting segmental defect repairs that does not have or greatly reduces the risks of disease transmission and negative immune response would be greatly appreciated in the art.

Distraction osteogenesis is yet another method of segmental defect repairs is currently used in the art. Distraction osteogenesis is the process by which an osteotomy is performed on a bone and the two ends are gradually separated to allow the mechanical induction of new bone growth in between the two bone segments. Distraction osteogenesis is a three part process, consisting of: latency, distraction, and consolidation. The latency period is the initial inflammatory phase of fracture healing, typically lasting from three to ten days. The periodic lengthening that follows is known as the distraction phase. The optimum distraction cycle is 1 mm/day in four increments of 0.25 mm throughout the day. After the prescribed amount of lengthening has been established, the last phase of consolidation can occur, which is the stabilization of the bone. This final phase tends to last the same amount of time as the distraction phase. While distraction osteogenesis reduces the chance of cellular morbitity in the patient and greatly reduces the chance of disease transmission that occurs in allografts, distraction osteogenesis introduces the chance of infection caused by external fixation devices. Common infections from the external fixation devices include pin track infections and infections from loosening of the fixation pins. Another drawback of distraction osteogenesis is the length of time it takes for the process to be completed. Therefore, a method of conducting segmental defect repairs that does not have or greatly reduces the risks infection and reduces the time period to complete the repair would be greatly appreciated in the art.

In addition to the current methods of regenerating segmental defects discussed above, tests have been conducted to use synthetic scaffolds or carriers along with demineralized bone matrix (“DBM”). DBM has been shown to stimulate hyaluronic acid accumulation and alkaline phosphatase activity, thereby increasing the rate of bone formation. DBM comprises type 1 collagen, fibronectin, bone sialoprotein (BSP), and bone morphogenic protein (BMP)-2, 4, and 7, and may be produced from human bone chips or obtained commercially, and has often been used as an adjunct factor to facilitate fracture healing. While DBM and other osteogenic compounds such as BMP have been used in conjunction with marrow aspirates or allograft to facilitate bone healing in segmental defect repair or in spine fusion, DBM falls short in regenerating segmental defects. Specifically, DBM lacks mechanical properties and is easily displaced in the large, unconfined space commonly seen in segmental defects, making it a poor choice for any segmental defect that occurs on a load bearing joint.

Commercial products, such as Grafton® by Osteotech, Inc., AlloMatrix® by Wright Medical Tech, attempt to address this issue by combining DBM with various molecular carriers such as glycerol, hyaluronic acid, procine gelatin, calcium sulfate, and Pluronic-F127 (an ethylene oxide/propylene oxide block copolymer). However, tests have shown that the lack of mechanical integrity of DBM, even when using these molecular carriers. In order to make up for this drawback, some have used DBM supported with additional non-degradable metallic hardware. For example, DBM and bone chips have been used with a titanium mesh cage to fit outside of the treated regeneration site to provide mechanical support to the DBM implant site. However, the use of such permanent metallic hardware can cause immune response in the patient. Therefore, a method of conducting segmental defect repairs that does not have or greatly reduces the use of permanent mechanical devices is desirable.

When the above treatment options fail, alternative treatment may involve serious consequences of leg shortening or amputation.

An indirect casting approach previously described by Chu et al. Biomaterials 28:459-467, 2007 was used for scaffold fabrication. For additional information on related techniques please see WO/2006-088866, which is incorporated herein by reference in its entirety. As previously reported, successful manufactured load-bearing tissue engineering scaffolds have been created and successfully used to induce bone regeneration in critical-sized segmental defects in rat femurs (Chu, et al., 2007). Tube-shaped scaffolds were manufactured from poly(propylene) fumarate/tricalcium phosphate (PPF/TCP) composites.

A biodegradable, load-bearing carrier for delivery of an osteogenic compound illustratively is made from high strength biodegradable composites. When first implanted, the carrier provides an initial biomechanically stable environment for bone formation across the interface between bone and carrier. The osteogenic compound-carrying biodegradable carrier may provide an osteoinductive environment to attract stem cells and progenitor cells to migrate to, populate, and mineralize on the carrier surface, illustratively to form a continuous bridge between the proximal and distal segments of the bone. Finally, the degradable carrier can degrade allowing the bone to fill in the space left by carrier. The bone may then continue to remodel to physiological geometry and mechanical properties.

As used herein, the term “carrier” or “structural carrier” refers to this high strength structural carrier to be used in conjunction with an osteogenic compound such as DBM. The DBM may be supplied in a molecule carrier (i.e. hyaluronic acid, glycerol, calcium sulfate hemihydrate, etc.). When referring to such molecule carrier, the term “molecule carrier” is used. While many of the examples use DBM, it is understood that other materials that provide for an osteoinductive environment may be used as well. Such materials include, but are not limited to, bone morphogenic protein (BMP) (including bone morphogenetic protein-2, bone morphogenetic protein-4, and bone morphogenetic protein-7), tissue growth factor beta (TGF-β), platelet-derived growth factor (PDGF), fibroblast growth factor (FGF), insulin-like growth factor, and vascular endothelial growth factor (VEGF). Moreover, the substances carried by the structural carrier are not limited to proteins. The structural carrier can include antibiotics or anti-inflammatory drugs. Suitable antibiotics include, but are not limited to, benzylpenicillin, cefazolin, clindamycin, vancomycin, nafcillin, and ciprofloxacin. The structural carrier can also include other substances to promote an osteoinductive environment including cells.

The use of DBM, BMP, or other osteogenic compounds in treating segmental defects may be combined with a high strength, load-bearing, and biodegradable carrier. When first implanted, the load-bearing carrier provides an initial biomechanically stable environment for bone formation across the bone scaffold junction. The osteogenic compound released from the biodegradable carrier then provides an osteoinductive environment to attract stem cells and progenitor cells to migrate to, populate, and mineralize on the scaffold surface, illustratively to form a continuous bridge between the proximal and distal segments of the bone. Finally, the degradable carrier does degrades and in time allows the patient's own bone to fill in the space left by carrier and continue to remodel to the physiological geometry and mechanical properties.

Healing gaps larger than the critical bone gap in animals is difficult. Fortunately, the use of mechanical structures, strategically placed within the gap has helped to treat these types of bone gaps in many mammals. In some of the approaches that have worked the mechanical structure placed in the gap is positioned so as to bear weight while allowing for bone regeneration. This achievement has rarely if ever been performed in the long bones of mice in the presence of growth factors alone (e.g. without the addition of cells). The exact reason for this is not known. Without being bound by any one theory, hypothesis or explanation it may that something unique to mouse physiology complicates or otherwise disfavours closing these sized gaps in mice. Still another possibility is the ratio of the defect length-to-bone length. Indeed, in the mouse a 4 mm segmental defect is required (to create a “critical sized defect” whereby the defect cannot heal on its own) and the length of mature mouse femurs is ˜10-12 mm, resulting in approximately a 1:3 ratio. While, in a rat which is a much larger animal a 5 mm segmental defect is sufficient to create a critical sized defect and as a rat femur is ˜40 mm, this results in approximate 1:8 ratio of gap to over all bone length. Similarly, the cross sectional area of a rat's femur is significantly larger than the cross sectional area of a mouse's femur. The greater cross sectional area of the bone segments bracketing the critical sized defect imply that there are more margins for error when contacting a scaffold or other inset in the bone gap in a rat then there is performing a similar procedure in a mouse. Due in part to its higher gap to bone length ratio and the greater cross sectional area of the rat femur an implant in a rat's bone gap is inherently more stable then is an implant in a corresponding gap in a mouse femur. Accordingly, it is possible that the additional stability in the rat, due to the increased support on each end of the femur allows for more flexibility in the scaffold design and surgical precisions. Whereas with the reduced support and stability in the mouse femur, the precision of scaffold design, scaffold structure position, and surgical technique is of heightened importance to promote bone gap closure. Near ideally positioning of a scaffold within a bone gap requires a scaffold engineered to almost perfectly fit into the bone gap. A less than optimal alignment of the scaffold, due perhaps to imperfections in the scaffold itself and the flanking bone tissue may result in movement of the scaffold and possible scaffold fracturing which will not allow adequate bone regeneration to close critical bone gaps in mice.

The inability to consistently promote bone growth sufficient to bridge critical sized gaps in mouse bone is especially unfortunate as mice are nearly ideal mammals for testing and developing of treatments, procedures and materials for treating bone defects, disease and injuries. The utility of mice as test subjects for such studies is due in part because so much is already known about their genetics and physiology. Additional factors that make these animals ideal candidates for such studies include their quick rate of growth, when using the same strain they serve as a fairly homogeneous study population, the wide variety of transgenic animals available to study and the relative ease with which new transgenic animals can be engineered.

Thus, one aspect of the present invention provides an implant scaffold for facilitating bone healing in a mouse. In some embodiments, the implant may comprise an osteogenic material, and a structural carrier formed from a biodegradable material, the structural carrier comprising a body and having at least one recess therein, wherein the structural carrier contains the osteogenic material in the recess. In various non-limiting illustrative embodiments, the osteogenic material may be selected from the group consisting of demineralized bone matrix and bone morphogenic protein; the body of the scaffold may form generally cylindrical shape. The scaffold body may further define a plurality of additional recesses; at least one of the additional recesses may be sized to receive a screw for plate fixation. In some embodiments, a second osteogenic material may be provided in one of the additional recesses, optionally wherein the osteogenic material and the second osteogenic material are different. In some embodiments, a first osteogenic material may be provided in a first time release material having a first time release profile the second osteogenic material is provided in a second time release material having a second different time release profile. In some embodiments, the first osteogenic material is VEGF and the second osteogenic material is BMP, and the first time release profile is a faster time release profile than the second time release profile; at least one of the recesses may comprises an antibiotic; the osteogenic material may be absorbed into an amount of dicalcium phosphate dihydrate (DCPD) cement that has been provided in the recess, the structural carrier may possess sufficient mechanical strength to be load bearing; and the biodegradable material may be poly(caprolacton) trimethacrylate/tricalcium phosphate (“PCLTMA/TCP”).

Still other aspect of the invention, an implant for facilitating bone healing in mice comprising an osteogenic material, and a structural carrier formed from a biodegradable material, the structural carrier comprising a wall extending from a first end to a second end and defining a central channel extending from an opening in the first end to an opening in the second end. In some embodiments the longitudinal center of the scaffold or other implant is hollow and can be used to accommodate at least one rod that is parallel to the length of the implant and extends beyond the first and second distal ends of the implant in to the first and second bone fragments that bracket the critical sized gap in the bone. Within the tubular wallchannel(s) or port(s) may form a generally cylindrical, elliptical, or tapered elliptical shape and the osteogenic material may be placed within at least one of the channels. In some embodiments the body of the scaffold or other implant includes a plurality of windows extending from the exterior side wall of the implant towards the longitudinal center of implant. Optionally wherein the osteogenic material is provided in one of the windows and a second osteogenic material is provided in another of the windows, wherein the osteogenic material and the second osteogenic material are different; at least one of the windows may comprise an antibiotic; the osteogenic material may be absorbed into an amount of dicalcium phosphate dihydrate (DCPD) cement that has been provided in one of the windows; the structural carrier may possess sufficient mechanical strength to be load bearing; and the biodegradable material may be PCLTMA/TCP. In some embodiments the osteogenic material may be selected from the group consisting of demineralized bone matrix and bone morphogenic protein.

Still other aspects of the invention include methods for repairing bone defects in mice. Some of these methods may comprise the steps of placing a structural implant comprising an osteogenic material into the defect site, and fixing the implant to surrounding bone tissue. Some of the methods may optionally include the step of leaving the implant in place and allowing the implant to degrade. Optionally, the fixing step may comprise using an intramedullary pin or may comprise using a plate and at least one screw.

Additional features of the present invention will become apparent to those skilled in the art upon consideration of the following detailed description of illustrative embodiments.

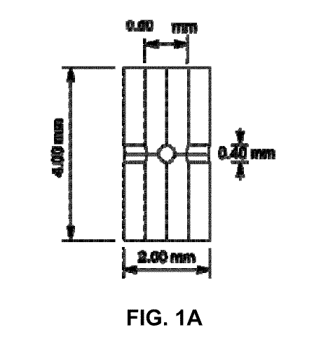

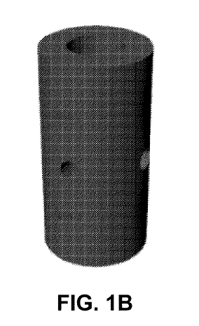

Referring now to inventive materials and methods used to successfully repair critical defects in mice. Dimensions of a representative Mouse femoral defect scaffold are provided in FIG. 1. Mouse femoral defect scaffolds were designed using commercially available CAD software (Rhinoceros, McNeel North America) to have a tubular geometry with an outer diameter of 2.0 mm, an inner diameter of 0.6 mm, and a height of 4.0 mm. These dimensions were determined to be appropriate based on careful measurements of mouse femurs. The scaffolds used in these examples include four side-holes each with a diameter of about 0.40 mm±0.05 mm. These holes could be used as drug depots within the scaffold.

### Experiment 1: Fabrication of Scaffold for Implantation in Mouse Femur

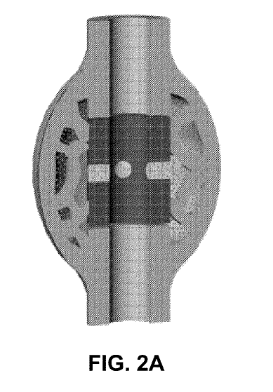

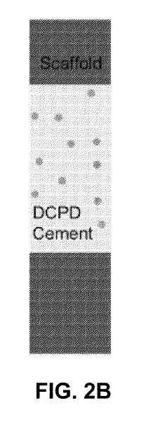

Referring now to FIGS. 2A and 2B. An indirect casting approach previously described by Chu et al. Biomaterials 28:459-467, 2007 was used for scaffold fabrication. Briefly, negative molds of the scaffolds were printed out of wax using a Solidscape T66 bench-top rapid prototyping machine. The molds were printed at a 38.1 μm layer thickness due to the small feature size. The scaffolds were made by casting a polymer-ceramic slurry comprising polypropylene fumarate) (PPF), N-vinyl pyrrolidinone (NVP), and β-tricalcium phosphate (β-TCP) into the molds. The mass ratio of PPF:NVP:β-TCP in the slurry was 5:3.75:3.3. Curing was achieved using the free radical initiator benzoyl peroxide, activated by N,N-dimethyl-p-toluidine. It is important to note that in the manufacturing of the rat scaffold a 1 paste system including the initiator and activator is used (benzoyl peroxide and N,N-dimethyl-p-toluidine). Unique to the mouse scaffold, a 2 paste system must be used along with an additional step to ensure that no or very few or only very small bubbles are entrained within the slurry. This is of critical importance when manufacturing scaffolds for use in mice as the wall of the device is very thin even a small number of bubbles of virtually any macroscopic size can compromise the biomechanical integrity of the scaffold wall.

In order to manufacture mouse scaffolds suitable for use in mice 2 separate pastes are created; One of the pastes includes a free radical initiator benzoyl peroxide while another paste includes N,N-dimethyl-p-toluidine. The individual pastes are then mixed together to ensure a more homogeneous mixture that includes few bubbles; after homogeneous mixing any or virtually all remaining bubbles are removed from the mixture by placing the mixture under vacuum for about 30 seconds (0.24 hp pump). The timing of the vacuum step is also critical as paste will cure (harden) in the bottle if it is left under vacuum for too long while too few bubbles will be removed from the paste if the time that the paste is placed under vacuum is too short. The wax molds were then removed by dissolution in acetone. Finally, the side-holes of the scaffolds were packed with dicalcium phosphate dihydrate (DCPD) cement, which served as the drug carrier. The DCPD cement was prepared using a 1:1 molar ratio mixture of monocalcium phosphate monohydrate and β-TCP, and deionized water as the mixing liquid. A powder:liquid mass ratio of 4:3 was used to prepare the cement, which resulted in a microporosity of approximately 55%. The scaffolds were stored in a vacuum dessicator chamber for 24 h prior to use in order to remove excess water from the DCPD cement and facilitate drug uptake by the cement.

Experiment 2: Implantation of a Scaffold within a Critical Sized Defect in a Mouse Femur

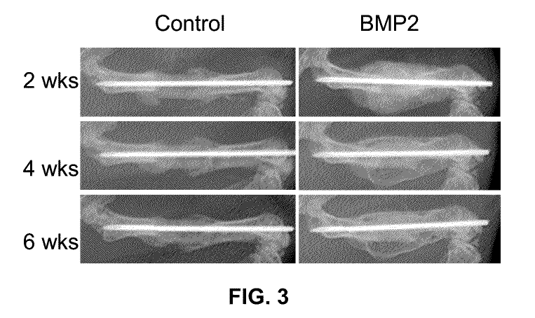

Referring now to FIG. 3. A critical sized femoral defect was created in the right femur of ten week old C57BL/6 male mice. One inventive scaffold was carefully inserted into the defect site. Control scaffolds contained no BMP-2. X-rays of femurs 2, 4 and 6 weeks postoperatively demonstrate callus formation by 2 weeks in mice with scaffolds containing BMP-2 whereas controls remain unbridged even at 6 weeks postoperatively. This latter observation confirms that a critical size defect was created and that the scaffold has sufficient biomechanical properties to maintain the segmental defect size. Finally, it shows that BMP-2 is successfully delivered to the defect site to induce bone formation.

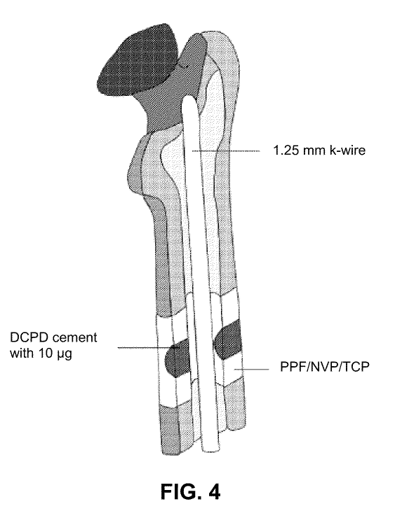

Referring now to FIG. 4. Dicalcium phosphate dehydrate (DCPD) was used as bone morphogenetic protein-2 (BMP-2) carrier. Twenty two scaffolds were implanted in 5 mm segmental defects in rat femurs stabilized with k-wire for 6 and 15 weeks with and without 10 μg of rhBMP-2. Bridging of the segmental defect was evaluated first radiographically and was confirmed by histology and micro-computer tomography (μ-CT) imaging. The scaffolds in the BMP group maintained the bone length throughout the duration of the study and allow for bridging. The scaffolds in the control group failed to induce bridging and collapsed at 15 weeks. Peripheral computed tomography (pQCT) showed that BMP-2 does not increase the bone mineral density in the callus. Finally, the scaffold in BMP group was found to restore the mechanical property of the rat femur after 15 weeks. These results demonstrated that the load-bearing BMP-2 scaffold can maintain bone length and allow successfully regeneration in segmental defects.

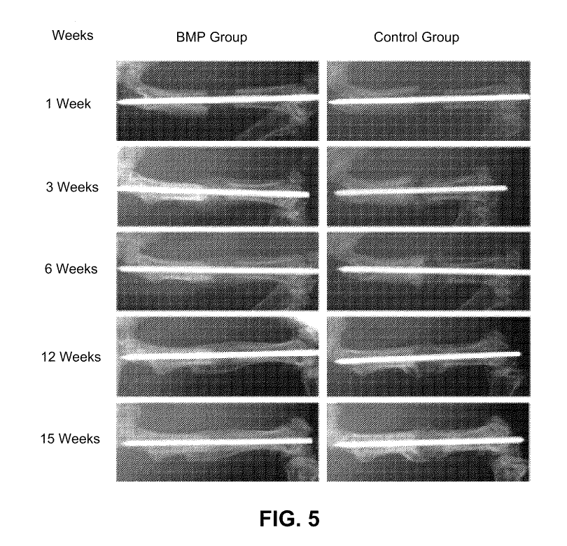

Referring now to FIG. 5. Representative serial radiological images of segmental defects in the BMP and control groups at one, three, six, 12 and 15 weeks post-operatively. At three weeks, callus had formed and bridged the segmental defect in the BMP group. In the control group, some cortical bone thickening and callus formation was evident immediately adjacent to the scaffold; however, there was no bridging callus. Between six and 15 weeks, the bridging callus in the BMP group showed signs of consolidation and remodeling. In contrast, in the control group only isolated regions of radio-opacity were evident within the defect region and no bridging callus was present.

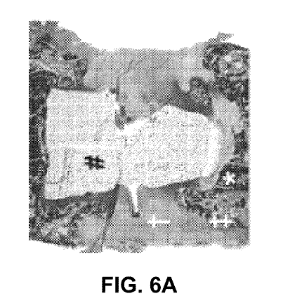

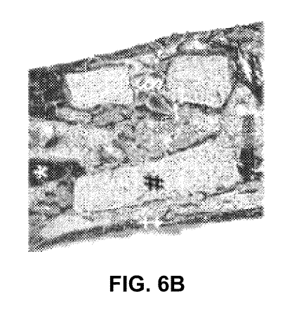

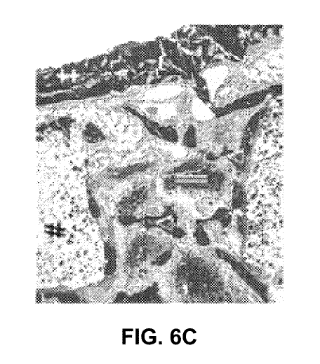

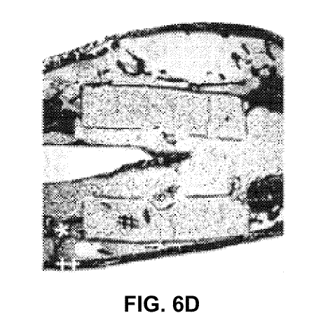

Referring now to FIG. 6. Representative histological images of segmental defects in the (A) control and (B,C) rhBMP groups at six weeks post-operatively. Sections are stained with McNeal's tetrachrome, which stains bone black. (A) Segmental defects in the control group demonstrated cartilaginous union, whereas (B) defects in the BMP group were bridged by mineralized callus that (C) invaded the side hole and was on the surface of the scaffold, indicating scaffold osteoconductivity. Inflammatory cells were not present in either scaffold group. (D) By 15-weeks post-operatively in the BMP group, the osteoconductivity of the scaffold is evident by the formation of new bone on its surfaces. *=original cortex of the femoral diaphysis, #=weight bearing biodegradable scaffold, †=cartilaginous tissue, ‡=mineralized callus, §=side hole within the scaffold, ∥=residual dicalcium phosphate dihydrate cement carrying rhBMP-2, Δ=mineralized callus within the side hole and on the surface of the scaffold.

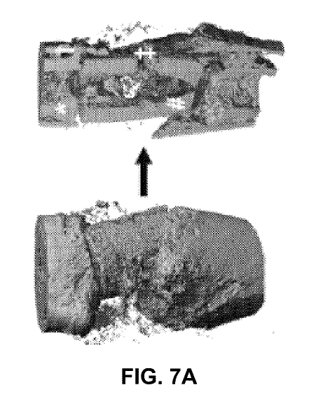

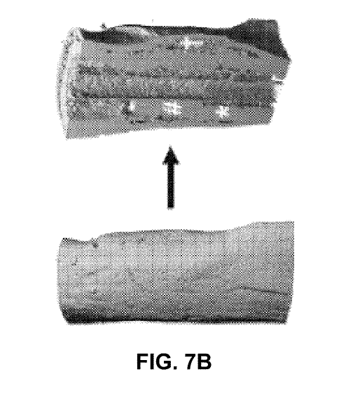

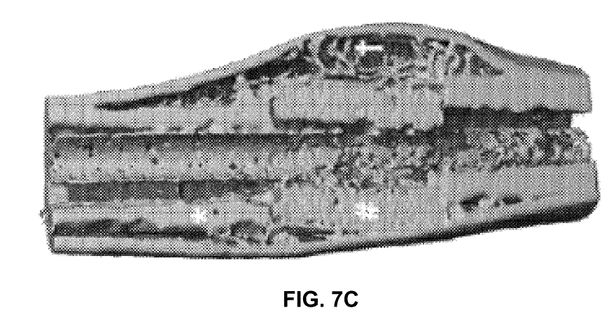

Referring now to FIG. 7. Representative external and cut-away images of segmental defects in the FIG. 7A control and FIG. 7B BMP groups, as assessed by microcomputed tomography at six weeks post-operatively. Referring now to FIG. 7A. Segmental defects in the control group had minimal bone surrounding the scaffold and the reparative callus did not bridge the defect. Referring now to FIG. 7B In contrast, the BMP group had a continuous mineralized callus around the scaffold, and bridging trabeculae beneath the cortical layer of the callus were integrated with the scaffold, indicating scaffold osteoconductivity.

Referring now to FIG. 7C. By 15-weeks post-operatively in the BMP-group, the bridging trabeculae had thickened and there is evidence of bone formation of bone on the surfaces of the scaffold, indicating scaffold osteoconductivity. *=original cortex of the femoral diaphysis, #=weight bearing biodegradable scaffold, †=mineralized callus, ‡=side hole within the scaffold.

### Experiment 3: Use of Scaffold and BMP to Prevent Critical Bone Gaps in Mice

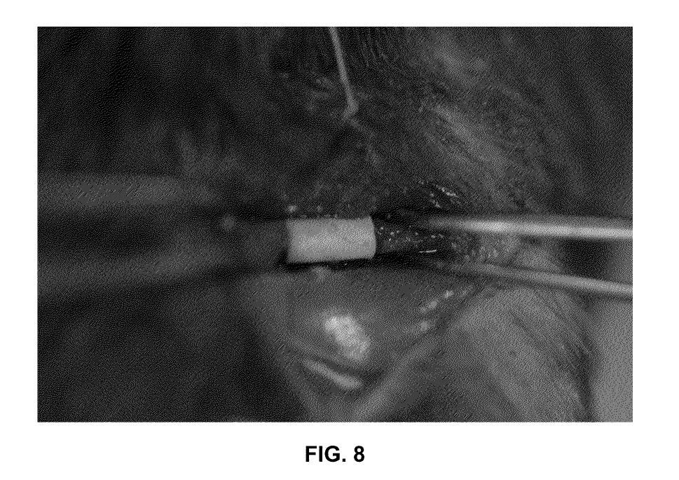

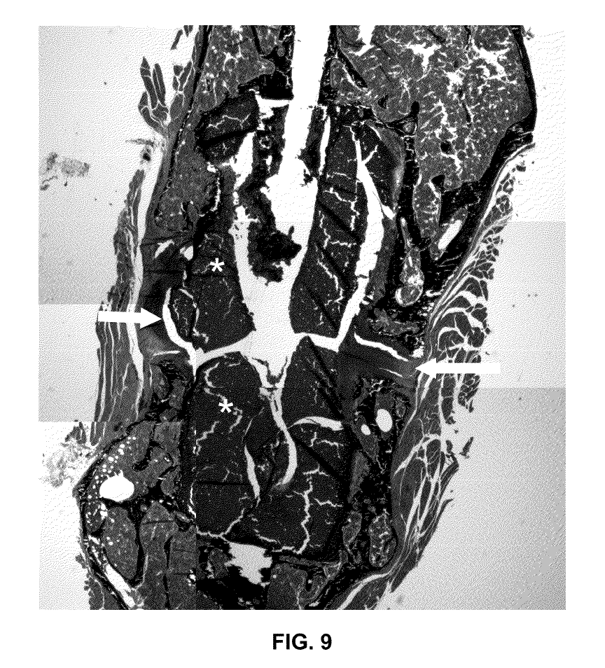

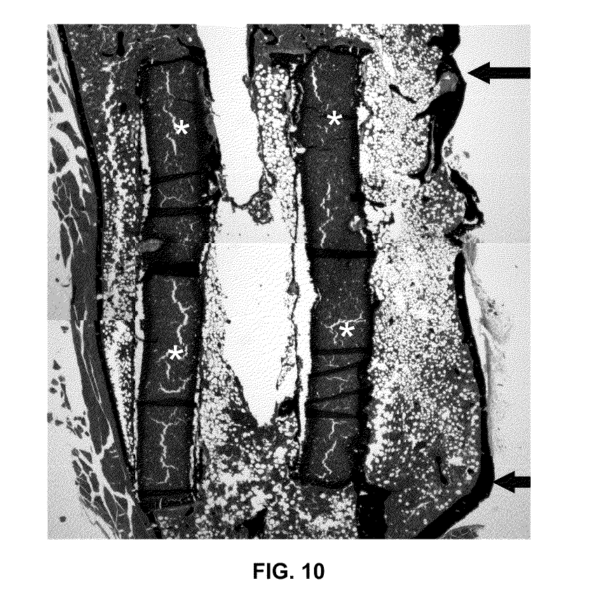

Referring now to FIGS. 3 and 8, 9 and 10. Mouse-size tube-shaped scaffolds were manufactured from poly(propylene) fumarate/tricalcium phosphate (PPF/TCP) composites (Chu, et al., 2007). Dicalcium phosphate dehydrate (DCPD) was used to carry bone morphogenetic protein-2 (BMP-2). Forty one scaffolds were implanted in 4 mm segmental defects in C57BL/6 mice and were stabilized with 27-gauge needle for 6 or 15 weeks with (n=27) and without (n=14) 4 μg of rhBMP-2 (FIG. 8.). Bridging of the segmental defect was evaluated using x-ray. At 2 weeks, the BMP-2 group starts to show callus formation. At 4-6 weeks, a complete bridging of the segmental defect occurred (FIG. 3.). FIGS. 9. and 10. are composite histological images of control (FIG. 9.) and BMP-2 (FIG. 10.) mouse scaffolds 15 weeks post-operative. Importantly, bridging callus is observed in mice containing BMP-2 treated scaffolds (FIG. 10.), whereas bridging is not evident in mice containing control scaffolds (FIG. 9.) The * in FIGS. 9 and 10 indicate the scaffold. The white arrows in FIG. 9 show the soft tissue between the two bone ends indicating non-union in the control group. The black arrows in FIG. 10. show the continuous callus between the two bone ends indicating successful bridging in the BMP-2 group.

Steps for increasing the efficiency with which the scaffolds are made and the quality of the scaffolds include method such as the following. Custom sized scaffolds can be manufactured using many of the techniques known in the art. For example, a biodegradable polymer, polypropylene fumarate (PPF) (Mn=1,750) may be used. A thermal-curable PPF/tricalcium phosphate (TCP) suspension can be prepared by mixing PPF and TCP powder. The tube shaped scaffold for insertion into the control group in a mouse femur may have an outer diameter of 2 mm and inner diameter of 0.6 mm, with four side openings of 400 μm in diameter on the side walls. The negative of the carrier design is generated using Boolean operations on a computer and it can be used as the mold design to make a compound casting mold. The 3D mold design can be sliced into 2D layers of 12.5 μm in thickness using commercial software such as SolidWorks®, Solidscape Inc. New Hampshire. The processed file is transferred to the 3-D Inkjet Printing Machine (T66, Solidscape Inc. NH). The mold design will be used to make the wax casting mold on 3-D Inkjet Printing Machine (T66, Solidscape Inc. NH). PPF/TCP composite slurry will be combined with 0.5 wt % of Benzoyl Peroxide (thermal initiator) and 10 μl of dimethyl p-toluidine (accelerator) just prior to casting. The slurry will be used to cast into the wax mold and allowed to solidify. After the polymerization reaction completed, the wax mold will be removed by solvent to reveal the PPF/TCP carrier. The four side windows will be filled with dicalcium phosphate dihydrate cements.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

1. An implantable scaffold for treating critical sized bone gaps in mice, comprising:
a biodegradable scaffold; said scaffold having a body, the body having sides; a first distal end and a second distal end, and a center wherein the center accommodates or includes an anchoring member that extends beyond both the first and the second distal ends of the body of the scaffold and wherein the scaffold is sized to fit into a critical sized gap in a mouse femur; and
at least one bio-reactive compound that promotes bone growth and or healing, wherein the compound is associated with the biodegradable scaffold.

2. The scaffold according to claim 1, wherein the anchoring member is a rod or wire (composed of metal, plastic or other material of sufficient strength and biocompatability).

4. The scaffold according to claim 1, wherein the anchoring member is substantially comprised of a biodegradable material.

5. The scaffold according to claim 1, wherein the body of the device is sized such that the first distal end and the second distal end of the scaffolding are in contact with surfaces of mouse femur bracketing a critical sized gap in a mouse femur.

6. The scaffolding according to claim 1, wherein the scaffold further includes at least one channel, the channel being located in a plane that intersects the center of the scaffold.

7. The scaffold according to claim 1, wherein the channel is of substantially uniform over its entire length.

8. The scaffold according to claim 1, wherein the channel is in the shape of a funnel.

9. The scaffold according to claim 1, wherein the channel extends from the center of the scaffold to at least one hole in the side of the scaffold.

10. The scaffold according to claim 6, wherein at least one channel in the scaffold includes at least one compound that promotes bone growth and or bone health.

11. The scaffold according to claim 1, wherein the compound that promotes bone growth is BMP-2.

12. The scaffold according to claim 1, wherein the bio-reactive compound is an antibiotic;

13. The scaffold according to claim 1, wherein the bio-reactive compound is included in a formulation that includes dicalcium phosphate dihydrate.

14. The scaffold according to claim 10, wherein at least one channel in said scaffold is packed with a formulation dicalcium phosphate dihydrate that includes BMP.

15. The scaffold according to claim 1, wherein the body of the scaffold is substantially comprised of N-vinyl pyrrolidinone and β-tricalcium phosphate.

16. The scaffold according to claim 15, wherein the body of the scaffold is free of macroscopic voids with the sides of the scaffold comprising the body of the scaffold and surrounding the center of the scaffold.

17. The scaffold according to claim 1, wherein the scaffold is in the shape of a cylinder and wherein the diameter of the cylinder is less than or equal to about 0.6 mm.

18. A method for manufacturing a biodegradable scaffold, comprising the steps of:
providing a first paste that include free radial initiator benzoyl peroxide;
supplying a second paste that includes N, N-dimethyl-p-toluidine; and
mixing the first and the second paste and exposing the mixture to a vacuum for at least 30 seconds.
